(19) United States
(12) Patent Application Publication
(10) Pub. No.: US 2026/0224854 A1
(43) Pub. Date: Aug. 6, 2026

(54) Catheter System With High-Strength Stabilization Anchor Securement

(71) Applicant: Becton, Dickinson and Company, Franklin Lakes, NJ (US)

(72) Inventor: Jonathan Karl Burkholz, Salt Lake City, UT (US)

(21) Appl. No.: 19/148,014

(22) PCT Filed: Jan. 15, 2024
(86) PCT No.: PCT/US2024/011536

§ 371 Date: Jul. 15, 2025

Related U.S. Application Data
(60) Provisional application No. 63/480,025, filed on Jan. 16, 2023.

(51) Int. Cl. A61M 25/06; A61M 25/02; A61M 39/02

(52) U.S. Cl. CPC A61M 25/0637; A61M 39/0247; A61M 2025/0253; A61M 2039/0258

(57) ABSTRACT

Provided herein is a catheter system including a catheter and associated catheter device, with the catheter device including a side port in fluid communication with the catheter. Extension tubing is provided that is in fluid communication with the side port, the extension tubing having a proximal access port. A force controlled release connector is coupled to the proximal access port and is separable therefrom when a predetermined separation force is applied. A stabilization anchor platform is spaced from the catheter device and positioned along the extension tubing, distal from the force controlled release connector, with an attachment member provided for securing the stabilization anchor platform relative to a skin surface of a patient. The attachment member retains the stabilization anchor platform in place relative to the skin surface with a securement force that is greater than the predetermined separation force of the force controlled release connector.

### CROSS-REFERENCE TO RELATED APPLICATION

The present application claims priority to U.S. Provisional Application Ser. No. 63/480,025 entitled “Catheter System with High-Strength Stabilization Anchor Securement” filed Jan. 16, 2023, the entire disclosure of which is hereby incorporated by reference in its entirety.

### BACKGROUND OF THE INVENTION

### Field of the Invention

The present disclosure relates to a catheter system with high-strength securement of a stabilization anchor.

### Description of Related Art

Catheters are commonly used to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

In some instances, the VAD is part of a larger catheter system, where tubing or an intravenous line extends out from an adapter or connector of the VAD to provide additional flexibility for medical professionals in terms of administering and/or withdrawing fluids from the VAD. The tubing may be part of an extension set that is integrated with the VAD (an integrated catheter system) or provided separate from the VAD (a non-integrated catheter system), according to various embodiments.

There are times when current VADs, and consequently extension sets or tubing, are manipulated manually to enable blood draw or introduction of a fluid; however, doing so in an uncontrolled way can increase incidence of dislodgement of the VAD from the vasculature, thus shortening the life of the catheter indwell. While securement devices exist and are sometimes employed for attaching the extension sets or tubing to the skin of a patient, such securement devices may not provide a sufficient securing strength needed to ensure that the VAD is retained in place. For example, the securing strength provided by the securement device may be less than a release strength of a controlled release connection provided at the proximal end of the extension set.

Accordingly, a need exists in the art for improved devices and methods for securing extension sets or tubing of a catheter system to a patient, so as to prevent accidental dislodgement of the VAD.

### SUMMARY OF THE INVENTION

Provided herein is a catheter system that includes a catheter device for receiving a catheter and including a side port in fluid communication with the catheter, the catheter device comprising a catheter adapter or catheter connector. The catheter system also includes extension tubing in fluid communication with the side port of the catheter device, the extension tubing having a proximal access port coupled to a proximal end portion thereof. The catheter system further includes a force controlled release connector coupled to the proximal access port, wherein at least a portion of the force controlled release connector is configured to separate from the proximal access port when a predetermined separation force is applied to the force controlled release connector. The catheter system still further includes a stabilization anchor platform spaced from the catheter device and positioned along the extension tubing, distal from the force controlled release connector, and an attachment member for securing the stabilization anchor platform relative to a skin surface of a patient. The attachment member retains the stabilization anchor platform in place relative to the skin surface with a securement force that is greater than the predetermined separation force that causes the force controlled release connector to separate from the proximal access port.

In some embodiments, the stabilization anchor platform includes a main body substantially defining a perimeter of the stabilization anchor platform and a retainer portion provided on the main body and configured to retain the extension tubing therein.

In some embodiments, the main body comprises a pair of stabilizing wings, and wherein the retainer portion is positioned between the pair of stabilizing wings.

In some embodiments, the attachment member is an upper securement member having an adhesive on at least a portion of a bottom surface thereof, the upper securement member positioned over the stabilization anchor platform such that the stabilization anchor platform is positioned between the skin surface and the upper securement member, and wherein a perimeter of the upper securement member is larger than the perimeter of the stabilization anchor platform, such that a first portion of the upper securement member adheres to the skin surface and a second portion of the upper securement member adheres to the stabilization anchor platform.

In some embodiments, a second portion of the upper securement member adheres to a top surface of the stabilization anchor platform, via the adhesive on the bottom surface of the upper securement member.

In some embodiments, the upper securement member includes slots formed therein through which the extension tubing is routed.

In some embodiments, the attachment member is an external stabilization device having an adhesive on a bottom surface thereof that adheres to the skin surface, the external stabilization device including a cut-out portion formed therein configured to receive the stabilization anchor platform, and wherein the external stabilization device includes another adhesive that secures the stabilization anchor platform within the cut-out portion.

In some embodiments, a shape of the cut-out portion matches a shape of the stabilization anchor platform.

In some embodiments, the attachment member includes both of the upper securement member and the external stabilization device.

In some embodiments, the attachment member is a pattern or layer of adhesive that adheres the stabilization anchor platform directly to the skin surface.

In some embodiments, the stabilization anchor platform includes one or more pass-through features passing through the stabilization anchor platform from a front surface of the stabilization anchor platform to a back surface of the stabilization anchor platform, the one or more pass-through features configured to transfer and distribute adhesive applied thereto front the front surface to the back surface.

In some embodiments, the attachment member includes both of the upper securement member and the pattern or layer of adhesive that adheres the stabilization anchor platform directly to the skin surface.

In some embodiments, the securement force is an adhering force of greater than 7 lbs, greater than 5 lbs, or greater than 3 lbs.

In some embodiments, the catheter system includes a clamp positioned along the extension tubing, wherein the stabilization anchor platform is positioned distal to the proximal access port and proximal to the clamp.

In some embodiments, the proximal access port is a needle-free connector.

In some embodiments, the force controlled release connector includes a disconnect connector and a release feature coupled to the needle-free connector of the proximal access port, the release feature is engaged with the disconnect connector and configured to be disengaged from the disconnect connector when the predetermined separation force is applied to the disconnect connector or the needle-free connector of the proximal access port.

In some embodiments, the catheter system includes a secondary stabilization anchor platform configured to stabilize the catheter device.

In some embodiments, the extension tubing is integral with the catheter adapter, as part of an integrated catheter system.

In some embodiments, the extension tubing and the catheter connector are coupleable to a catheter hub connected to the catheter, as part of a non-integrated catheter system.

Also provided herein is a method for securing the catheter system to the skin surface of a patient, with the method including adhering the stabilization anchor platform in place relative to the skin surface via the attachment member, with the attachment member adhered to the skin surface with the securement force, which is greater than the predetermined separation force that causes the force controlled release connector to separate from the proximal access port.

### BRIEF DESCRIPTION OF THE DRAWINGS

FIG. 1 is a top view of an integrated catheter system, according to an aspect or embodiment of the present application;

FIG. 2 is a top view of a non-integrated catheter system, according to an aspect or embodiment of the present application;

FIG. 3 is top view of a force controlled release connection included in the system of FIG. 1 and/or FIG. 2, according to a further aspect or embodiment of the present application;

FIG. 4 is cross-sectional view of the force controlled release connection of FIG. 3, with the connection in a disconnected state, according to a further aspect or embodiment of the present application;

FIG. 5 is a perspective view of a stabilization anchor platform and attachment member included in the system of FIG. 1 and/or FIG. 2, according to an aspect or embodiment of the present application;

FIG. 6 is a perspective view of a stabilization anchor platform and attachment member included in the system of FIG. 1 and/or FIG. 2, according to an aspect or embodiment of the present application;

FIG. 7 is a perspective view of a stabilization anchor platform and attachment member included in the system of FIG. 1 and/or FIG. 2, according to an aspect or embodiment of the present application;

FIG. 8 is a perspective view of a stabilization anchor platform and attachment member included in the system of FIG. 1 and/or FIG. 2, according to an aspect or embodiment of the present application; and

FIG. 9 is a perspective view of a stabilization anchor platform and attachment member included in the system of FIG. 1 and/or FIG. 2, according to an aspect or embodiment of the present application.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

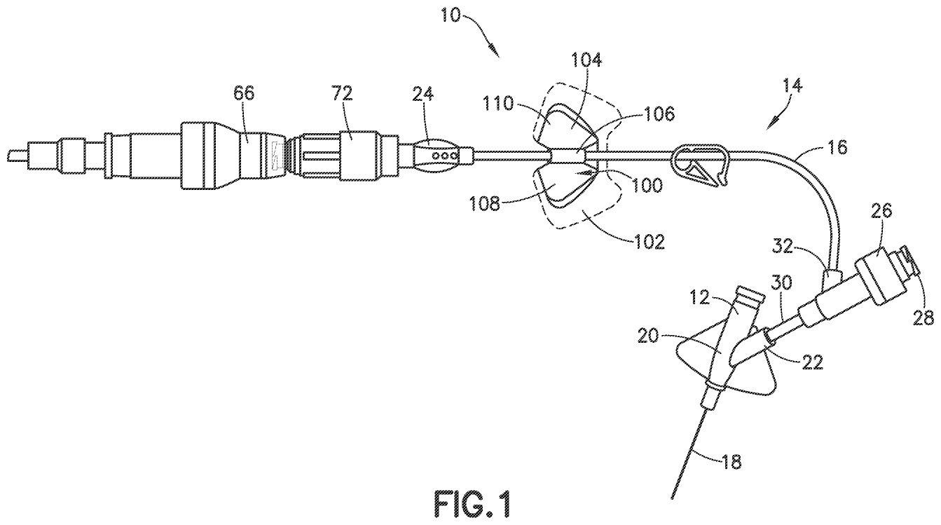

Referring to FIG. 1, an integrated catheter system 10 is shown, according to one aspect or embodiment with which embodiments of the invention may be implemented. The integrated catheter system 10 includes a catheter device 12 in the form of a catheter adapter (hereafter “catheter adapter 12”), along with an integrated extension set 14 (including extension tubing 16). The catheter adapter 12 includes a catheter 18, a body 20 receiving the catheter 18, and a side port 22 in fluid communication with the catheter 18. The integrated extension set 14 includes a proximal access port 24 coupled to a proximal end portion of the extension tubing 16, with the extension tubing 16 in fluid communication with the side port 22 of the catheter adapter 12.

In some aspects or embodiments, the system 10 may include a near patient access port 26 in fluid communication with the side port 22 of the catheter adapter 12, with the near patient access port 26 including a connector portion 28 configured to be coupled to a peripheral probe device. The near patient access port 26 is configured to provide catheter access to peripheral devices such as, e.g., a blood draw device (e.g., PIVO™ from Becton, Dickinson and Company), or a vascular access probe (VAP) for in-vein digital measurement of patient data such as temperature, pH, lactate, and/or other blood-based measurements. In some embodiments, the near patient access port 26 is coupled to the side port 22 of the catheter adapter 12 via a length of intermediate tubing 30. However, in other embodiments, the near patient access port 26 may be coupled directly to the side port 22, or connected via another intermediate member.

The near patient access port 26 includes the connector portion 28, which in some embodiments, is configured to be compatible with peripheral devices such as blood draw devices and/or vascular access probes. In some embodiments, the connector portion 28 is configured as a needle-free connector (NFC) configured to receive a blunt introducer of a blood draw device. More specifically, the connector portion 28 may be configured as a split-septum NFC with direct probe access such as, e.g., Q-Syte™ or SmartSite™M NFCs from Becton, Dickinson and Co., or any other appropriate split-septum NFC. Alternatively, in other embodiments, the connector portion 28 may be formed of a non-split-septum-type NFC. Furthermore, in some embodiments, the near patient access port 26 may include anti-microbial and/or flush-promoting features. For example, the near patient access port 26 may include one or more of an offset tubing port vortex-creating feature, a proximal flow-diverting feature, anti-microbial NFC lubricant, anti-microbial eluting surface coating(s) or insert(s), etc. With the near patient access port 26 fluidly coupled to the catheter adapter 12 via the side port 22, the system 10 provides for probe (or tube) access from a peripheral probe device through the indwelling catheter 18. The near patient access port 26 further includes a secondary port 32 positioned near a distal end thereof. In some embodiments, the secondary port 32 is coupled to the extension tubing 16 of integrated extension set 14. A clamp 34 may be provided on the integrated extension set 14, with the clamp 34 configured to selectively restrict flow through the extension tubing 16 of integrated extension set 14.

In other aspects or embodiments, extension tubing 16 of the integrated extension set 14 may extend directly from the side port 22 of the catheter, with the system 10 not including the near patient access port 26. In such embodiments, the system 10 may incorporate the BD Nexiva™ closed peripheral IV catheter system or the BD Nexiva™ Diffusics™ closed IV catheter system, as non-limiting examples.

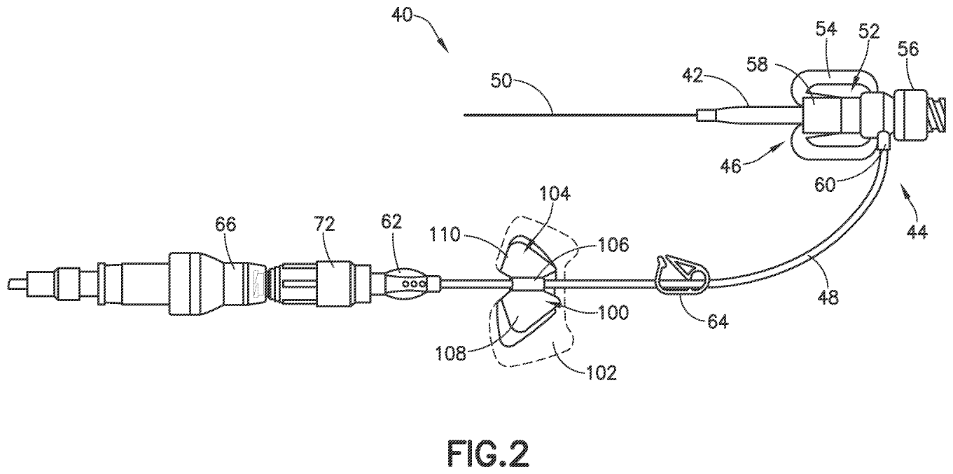

Referring to FIG. 2, a non-integrated catheter system 40 is shown, according to another aspect or embodiment with which embodiments of the invention may be implemented. The non-integrated catheter system 40 includes a catheter hub 42 and a non-integrated extension set 44 that includes a catheter device 46 in the form of a catheter stabilizing catheter connector (hereafter “catheter connector 46”), along with extension tubing 48. The catheter hub 42 receives a catheter 50 and is coupled to the catheter connector 46. In one aspect or embodiment, the catheter hub 42 is the AccuCath™ catheter system commercially available from Becton, Dickinson and Company.

The catheter connector 46 is configured to be placed in contact with the skin surface of a patient at or near an insertion site of catheter 50. The catheter connector 46 can be any suitable shape, size, and/or configuration. In the illustrated non-limiting embodiment, the catheter connector 46 has a connector portion 52 and a stabilization portion 54. The connector portion 52 has a proximal port with a coupler 56, a distal port with a distal coupler 58, and defines at least one lumen (not shown) extending through or otherwise in fluid communication with the couplers 56 and 58. The proximal coupler 56 and/or the distal coupler 58 can be, for example, male or female luer locks and/or any other suitable coupler. The proximal coupler 56 can be configured as an NFC or the like and can be physically and fluidically coupled to peripheral devices such as, e.g., a blood draw device (e.g., PIVO™ from Becton, Dickinson and Company), or a vascular access probe (VAP) for in-vein digital measurement of patient data such as temperature, pH, lactate, and/or other blood-based measurements. The distal coupler 58 can be physically and fluidically coupled to catheter hub 42 such that the lumen of the connector portion 52 is at least selectively in fluid communication with the catheter 50. In some embodiments, an inner surface of the connector portion 52 can be configured to provide alignment, guidance, centering, etc., to an object or device (e.g., a blood draw catheter or the like, as described above) being advanced therethrough.

The connector portion 52 also includes and/or defines one or more additional ports, such as a side port 60. Side port 60 can be included in and/or can be a part of the connector portion 52, the proximal coupler 56, the distal coupler 58, and/or a combination thereof. For example, side port 60 may be distal to the proximal coupler 56. In other embodiments, however, a connector portion 52 can include and/or define a side port 60 that is formed by a structure or feature forming a proximal coupler. In some instances, positioning side port 60 in a desired position along a length of the connector portion 52, between the couplers 56 and 58, can allow for a reduced length of the connector portion 52 and/or can facilitate flushing and/or fluid transfer via side port 60.

Side port 60 defines a lumen (not shown) that is in fluid communication with the lumen between distal coupler 58 and proximal coupler 56. In other words, the connector portion 52 and/or the side port 60 can include and/or define a first lumen (e.g., between distal coupler 58 and proximal coupler 56) and a second lumen (e.g., lumen defined by side port 60 and extension tubing 48). As such, side port 60 can provide access to the lumen between distal coupler 58 and proximal coupler 56, which in turn can provide access to a device (e.g., a VAD) that is coupled to the distal coupler 58 and/or can provide access to a portion of the body in which the VAD is at least partially disposed.

In some embodiments, the arrangement of side port 60 can be such that the connector portion 52 forms, for example, a Y-connector or a T-connector. More particularly, side port 60 can be disposed substantially perpendicular (e.g., about 90 degrees) to the lumen of the connector portion 52 and near or adjacent the proximal coupler 56. In non-limiting embodiments, side port 60 extends from connector portion 52 at an angle, relative to the lumen between distal coupler 58 and proximal coupler 56, that is not 90 degrees (e.g., side port 60 extends at an angle of, for example and without limitation, 15-165 degrees, with all values and subranges therebetween inclusive). In non-limiting embodiments, more than one side port 60 is included in connector portion 52.

As shown in FIG. 2, side port 60 may be coupled to extension tubing 48 such that the extension tubing 48 is in fluid communication with the lumen of the side port 60. In some embodiments, the side port 60 and/or the extension tubing 48 can be and/or can form at least a portion of a fluid line that can be used to deliver fluid, remove fluid, flush fluid, and/or the like. In such embodiments, for example, an arrangement in which side port 60 is disposed adjacent to the proximal coupler 56 can enable flushing of the proximal coupler 56, any valve(s) included therein, and/or a space between any valve(s) and an inner surface of the connector portion 52 (e.g., defining at least a portion of the lumen).

As shown, the extension set 44 includes a proximal access port 62 coupled to a proximal end portion of the extension tubing 48, with the extension tubing 48 in fluid communication with the side port 60 of the catheter connector 46. A clamp 64 may be provided on the extension tubing 48, with the clamp 64 configured to selectively restrict flow through the extension tubing 48.

In each of the systems of FIGS. 1 and 2, a force controlled release connector 66 may be coupled to the proximal access port 24, 62. At least a portion of the force controlled release connector 66 is configured to separate from the proximal access port 24, 62 when a predetermined separation force is applied to the force controlled release connector 66. In some embodiments, the predetermined separation force that causes the force controlled release connector 66 to separate from the proximal access port 24, 62 is a force of approximately 5 lbs, as a non-limiting example, although it is recognized that the force controlled release connector 66 could have a greater or lesser separation force. At least a portion of the force controlled release connector 66 is thus configured to detach from the proximal access port 24, 62 to prevent excessive forces from being transmitted to the catheter adapter 12 (FIG. 1) or catheter connector 46 (FIG. 2) via the extension tubing 16, 48. For example, a line from an IV bag may be connected to the extension tubing 16, 48 via the force controlled release connector 66 such that excessive movement of the IV bag and line will merely detach the connection between the proximal access port 24, 62 and the force controlled release connector 66 rather than transferring that movement to the extension tubing 16, 48 and disrupting the placement of the catheter 18, 50.

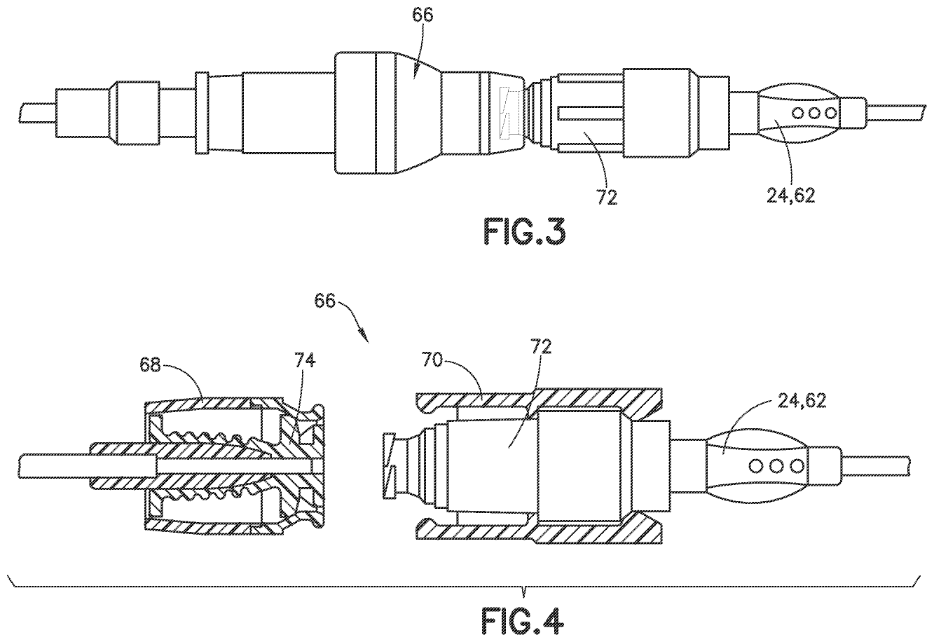

Referring to FIGS. 3 and 4, the force controlled release connector 66 is shown in greater detail according to one embodiment. The force controlled release connector 66 includes a disconnect connector 68 and a release feature 70 coupled to a needle-free connector 72 of the proximal access port 24, 62. The release feature 70 is engaged with the disconnect connector 68 and configured to be disengaged from the disconnect connector 68 when a predetermined separation force is applied to the disconnect connector 68 or the needle-free connector 72 of the proximal access port 24, 62. The disconnect connector 68 includes a valve member 74, which is configured to seal the disconnect connector 68 when separated from the needle-free connector 72. In one aspect or embodiment, the release feature 70 is configured to be biased radially outward when the predetermined separate force is reached. The release feature 70 and the disconnect connector 68 may form a camming arrangement where an axial force causes the release feature 70 to move radially outward, thereby releasing the disconnect connector 68 from the release feature 70. The release feature 70 may be formed integrally with the needle-free connector 72 or may be a separate component attached or coupled to the needle-free-connector 72 or the proximal access port 24, 62.

Referring again now to FIGS. 1 and 2, in each of the systems 10, 40, a stabilization anchor platform 100 is provided that is spaced from the catheter adapter 12 (FIG. 1) or catheter connector 46 (FIG. 2) and is positioned along the extension tubing 16, 48. The stabilization anchor platform 100—along with an accompanying attachment member 102 (generally shown in phantom in FIGS. 1 and 2, but the structure of which may vary according to various embodiments, as described in detail here after)—is configured to provide a primary securement point to a skin surface of a patient and minimize catheter dislodgement, infiltration, or catheter movement that may result in a tip of the catheter 18, 50 from being pulled out of a vein or artery. The stabilization anchor platform 100 is configured to isolate externally applied forces from the catheter adapter 12 or catheter connector 46 and the insertion site to limit movement of the catheter 18, 50 and/or dislodgement of the catheter 18, 50 due to forces being applied to an intravenous line or extension sets.

According to embodiments, the stabilization anchor platform 100 is generally composed of a substantially flat or planar main body 104 and a retaining section 106 (e.g., cylindrical passage) configured to secure the extension tubing 16, 48 therein. In some embodiments, the main body 104 includes a pair of stabilizing wings 108, 110 between which the retaining section 106 is centered, although other suitable arrangements may be utilized, including the main body 104 of stabilization anchor platform 100 being oval, triangular, round, square, rectangular, trapezoidal, or another suitable shape. The stabilization anchor platform 100 may be flexible, soft, semi-rigid, or rigid and may include features or a living hinge to promote flexibility for patient comfort. The stabilization anchor platform 100 may also include one or more features to improve attachment to the attachment member 102, such as protrusions, surface roughness, pockets, etc. The stabilization anchor platform 100 may include one or more colors or visual marking to provide catheter gauge, length, type, or procedure related information.

In some aspects or embodiments, the stabilization anchor platform 100 is positioned distal to the proximal access port 24, 62 and proximal to the clamp 34, 64. In other aspects or embodiments, the stabilization anchor platform 100 is positioned distal to the clamp 34, 64.

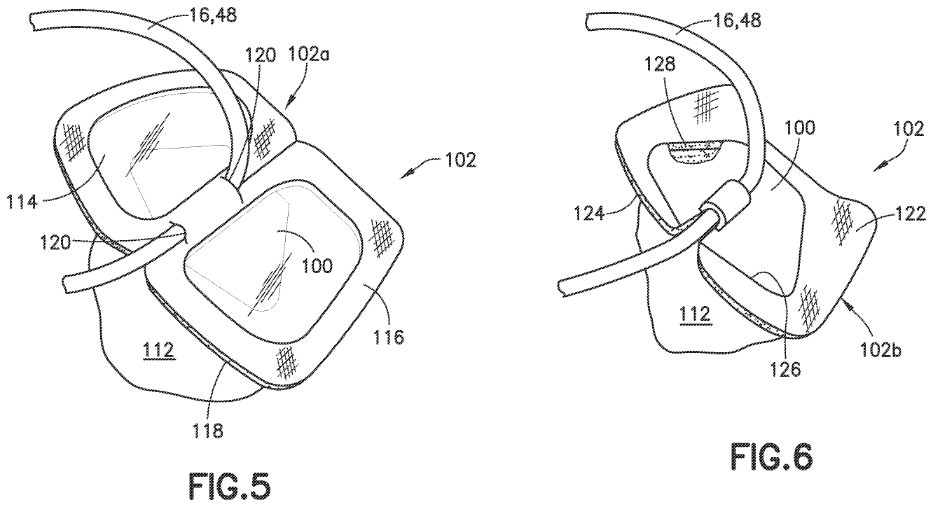

FIGS. 5-9 illustrate embodiments of various attachment members 102 that may be used to secure the stabilization anchor platform 100 in place relative to a skin surface (indicated by 112) of a patient. According to aspects of the invention, the attachment member(s) 102 are configured to apply a securing force (that secures the stabilization anchor platform 100 in place) that is greater than the predetermined separation force that causes the force controlled release connector 66 to separate from the proximal access port 24, 62. In some embodiments, the securement force is an adhering force that is at least 0.25 lbs greater than the predetermined separation force that causes the force controlled release connector 66 to separate from the proximal access port 24, 62. Thus, in a non-limiting example of where the predetermined separation force of the force controlled release connector 66 is 5 lbs, the adhering force of the attachment member(s) 102 should be 5.25 lbs or greater. According to non-limiting embodiments, and based on the predetermined separation force of the particular force controlled release connector 66 that is employed, the adhering force of the attachment member(s) 102 could be greater than 7 lbs, greater than 5 lbs, greater than 3 lbs, etc. By providing attachment member(s) 102 with a securing force that is sufficiently greater than the predetermined separation force of force controlled release connector 66, it is ensured that the stabilization anchor platform 100 is held in place until the force controlled release connector 66 releases, thereby preventing excessive movement or removal of the catheter 18, 50.

Referring first to FIG. 5, an attachment member 102 for securing the stabilization anchor platform 100 in place relative to a skin surface of a patient is provided in accordance with one aspect or embodiment. The attachment member 102 is provided as an upper securement member 102a that is applied over the stabilization anchor platform 100, such that the stabilization anchor platform 100 is positioned between the skin surface 112 and the upper securement member 102a. The upper securement member 102a may be configured as a top dressing or adhesive tape for securement. In one embodiment, the upper securement member 102a may include a transparent dressing portion 114 and a fabric bordered portion 116. The upper securement member 102a may come in a variety of shapes and sizes and, in the illustrated embodiment, has a shape generally similar to that of stabilization anchor platform 100 (i.e., a pair of wings) but has a perimeter and surface area that is larger than the perimeter and surface area of the stabilization anchor platform 100. The upper securement member 102a includes an adhesive 118 on a bottom surface thereof, such that at least a first portion of the upper securement member 102a (i.e., around the perimeter, such as the fabric bordered portion 116) adheres to the skin surface 112 and, optionally, a second portion of the upper securement member 102a (i.e., part of transparent dressing portion 114) adheres to a top surface of stabilization anchor platform 100.

In some embodiments, the upper securement member 102a includes one or more slots 120 formed therein that allows for passage of the extension tubing 16, 48 therethrough. That is, with the stabilization anchor platform 100 covered by the upper securement member 102a, it is necessary for the upper securement member 102a to accommodate routing of the extension tubing 16, 48 through the slots 120 so as to enable the extension tubing 16, 48 to enter and exit the area covered by the upper securement member 102a.

Referring now to FIG. 6, an attachment member 102 for securing the stabilization anchor platform 100 in place relative to a skin surface 112 of a patient is provided in accordance with another aspect or embodiment. The attachment member 102 is provided as a bottom securement member 102b that, in some embodiments, is an external stabilization device (hereafter “external stabilization device 102b”). The external stabilization device 102b includes, for example, a tricot base pad 122 having an adhesive 124 applied on the bottom surface thereof to attach to the skin surface 112 of the patient. A cut-out 126 is formed in the base pad 124 having a shape and perimeter/area that matches a shape and perimeter/area of the stabilization anchor platform 100. An adhesive 128 is applied in the area of cut-out 126 that secures the stabilization anchor platform 100 within the cut-out 126, so as to secure the stabilization anchor platform 100 relative to the external stabilization device 102b and to the skin surface 112. The adhesive 128 in the area of cut-out 126 may both secure a perimeter of the stabilization anchor platform 100 within the cut-out 126 and, optionally, a bottom of stabilization anchor platform 100 (adjacent the perimeter thereof) to the skin surface 112.

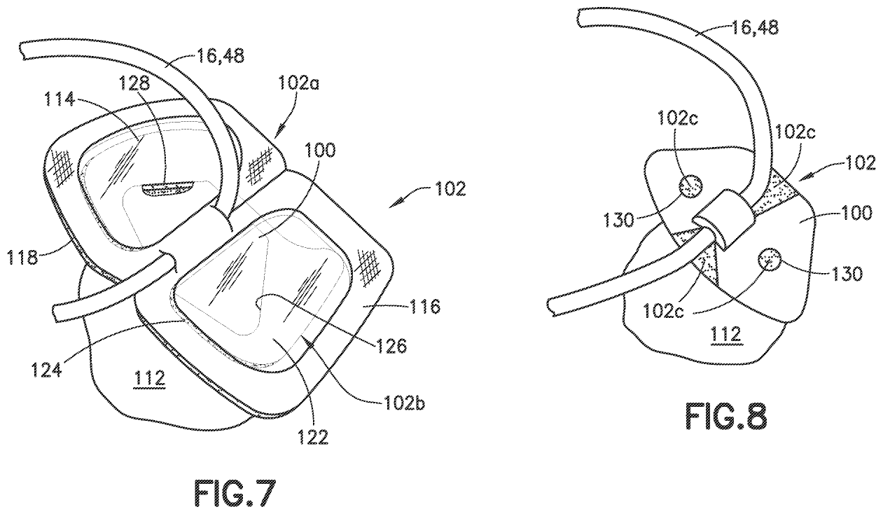

Referring now to FIG. 7, an attachment member 102 for securing the stabilization anchor platform 100 in place relative to a skin surface 112 of a patient is provided in accordance with another aspect or embodiment. The attachment member 102 is provided as both an upper securement member 102a and a bottom securement member 102b (i.e., external stabilization device 102b). As previously described, the upper securement member 102a may be configured as a top dressing or adhesive tape and, in one embodiment, the upper securement member 102a may include a transparent dressing portion 114 and a fabric bordered portion 116. The external stabilization device 102b may include a base pad 124 having a cut-out 126 formed therein having a shape and perimeter/area that matches a shape and perimeter/area of the stabilization anchor platform 100.

In arranging the upper and bottom securement members 102a, 102b relative to the stabilization anchor platform 100 and in securing the securement members and stabilization anchor platform 100 to a patient, the bottom securement member 102b (external stabilization device 102b) is attached to the skin surface 112 of the patient via an adhesive 124 applied on the bottom surface of the external stabilization device 102b. The stabilization anchor platform 100 is positioned within the cut-out 126 of the base pad 124 of the external stabilization device 102b and adhesive 128 is applied in the area of cut-out 126 to secure the stabilization anchor platform 100 within the cut-out 126. Upon the external stabilization device 102b and stabilization anchor platform 100 positioned and secured in place relative to the skin surface 112, the upper securement member 102a is then applied over the external stabilization device 102b and stabilization anchor platform 100. As shown in FIG. 6, the upper securement member 102a is sized such that the perimeter thereof extends out past the perimeter of the external stabilization device 102b. The upper securement member 102a includes an adhesive 118 on a bottom surface thereof, such that at least a first portion of the upper securement member 102a (i.e., around the perimeter, such as a fabric bordered portion 116) adheres to the skin surface 112 and, optionally, a second portion of the upper securement member 102a adheres to a top surface of the external stabilization device 102b and/or stabilization anchor platform 100. Accordingly, the upper securement member 102a further secures the stabilization anchor platform 100 (and external stabilization device 102b) in place relative to the skin surface 112.

Referring now to FIG. 8, an attachment member 102 for securing the stabilization anchor platform 100 in place relative to a skin surface 112 of a patient is provided in accordance with another aspect or embodiment. The attachment member 102 is provided as a pattern or layer of skin adhesive 102c that secures the stabilization anchor platform 100 directly to the skin surface 112. According to embodiments, the stabilization anchor platform 100 is a skin adhesive-compatible platform that may include features 130 thereon that allow dispensing of the skin adhesive 102c on the top of the stabilization anchor platform 100 and that facilitate distribution of the skin adhesive 102c to the bottom surface of the stabilization anchor platform 100 (i.e., “pass-through” features), such that the bottom surface of the stabilization anchor platform 100 may be securely attached to the skin surface 112. In other embodiments, skin adhesive 102c may be applied directly to the bottom of the stabilization anchor platform 100 and/or to the skin surface 112. In any of the above-described embodiments, the stabilization anchor platform 100 is pressed against the skin surface 112 (e.g., by the clinician or by a tape or dressing) and allowed to dry, to ensure formation of a strong adhesive bond.

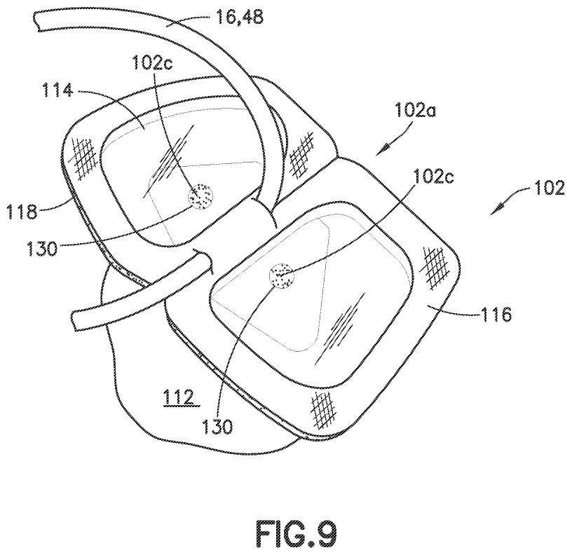

In some embodiments, and as shown in FIG. 9, the stabilization anchor platform 100 may be secured in place relative to the skin surface 112 via a combination of skin adhesive 102c between the stabilization anchor platform 100 and the skin surface 112 and an upper securement member 102a applied over the stabilization anchor platform 100. As previously described, the upper securement member 102a may be configured as a top dressing or adhesive tape and, in one embodiment, the upper securement member 102a may include a transparent dressing portion 114 and a fabric bordered portion 116.

In arranging and securing the stabilization anchor platform 100 and upper securement member 102a to a patient, skin adhesive 102c is first applied to the stabilization anchor platform 100. According to embodiments, skin adhesive 102c may be applied to pass-through features 130 on the top of the stabilization anchor platform 100 that distribute the adhesive to the bottom surface of the stabilization anchor platform 100, or skin adhesive 102c may be applied directly to the bottom of the stabilization anchor platform 100 and/or to the skin surface 112. Upon the stabilization anchor platform 100 being secured in place relative to the skin surface 112, the upper securement member 102a is then applied over the stabilization anchor platform 100. As shown in FIG. 9, the upper securement member 102a is sized such that the perimeter thereof extends out past the perimeter of the stabilization anchor platform 100. The upper securement member 102a includes an adhesive 118 on a bottom surface thereof, such that at least a first portion of the upper securement member 102a (i.e., around the perimeter, such as a fabric bordered portion 116) adheres to the skin surface 112 and, optionally, a second portion of the upper securement member 102a adheres to a top surface of the stabilization anchor platform 100. Accordingly, the upper securement member 102a further secures the stabilization anchor platform 100 in place relative to the skin surface 112.

In each of the embodiments described above in FIGS. 5-9, the attachment member(s) 102 are configured to apply a securing force (that secures the stabilization anchor platform 100 in place) that is greater than the predetermined separation force that causes the force controlled release connector 66 to separate from the proximal access port 24, 62 (FIGS. 1 and 2). By providing attachment member(s) 102 with a securing force that is greater than the predetermined separation force of force controlled release connector 66, it is ensured that the stabilization anchor platform 100 is held in place until the force controlled release connector 66 releases, thereby preventing excessive movement or removal of the catheter 18, 50.

Beneficially, embodiments of the disclosure provide a stabilization anchor platform and associated attachment member(s) positionable on extension tubing of a catheter system, with the stabilization anchor platform configured to provide a securement point to a skin surface of a patient and minimize catheter dislodgement, infiltration, or catheter movement that may result in a tip of the catheter from being pulled out of a vein or artery. The stabilization anchor platform is configured to isolate externally applied forces from a device (i.e. catheter adapter or catheter connector) coupled to the catheter (or catheter hub), at the insertion site, to limit movement of the catheter and/or dislodgement of the catheter due to forces being applied to an intravenous line or extension sets. For a catheter system with a force controlled release connector coupled to a proximal access port of the extension tube, the attachment member is configured to retain the stabilization anchor platform in place relative to the skin surface with a securement force that is greater than a predetermined separation force that causes the force controlled release connector to separate from the proximal access port.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A catheter system comprising:
a catheter device for receiving a catheter and including a side port in fluid communication with the catheter, the catheter device comprising a catheter adapter or catheter connector;
extension tubing in fluid communication with the side port of the catheter device, the extension tubing having a proximal access port coupled to a proximal end portion thereof;
a force controlled release connector coupled to the proximal access port, wherein at least a portion of the force controlled release connector is configured to separate from the proximal access port when a predetermined separation force is applied to the force controlled release connector;
a stabilization anchor platform spaced from the catheter device and positioned along the extension tubing, distal from the force controlled release connector; and
an attachment member for securing the stabilization anchor platform relative to a skin surface of a patient;
wherein the attachment member retains the stabilization anchor platform in place relative to the skin surface with a securement force that is greater than the predetermined separation force that causes the force controlled release connector to separate from the proximal access port.

2. The system of claim 1, wherein the stabilization anchor platform comprises:
a main body substantially defining a perimeter of the stabilization anchor platform; and
a retainer portion provided on the main body and configured to retain the extension tubing therein.

3. The system of claim 2, wherein the main body comprises a pair of stabilizing wings, and wherein the retainer portion is positioned between the pair of stabilizing wings.

4. The system of claim 2, wherein the attachment member comprises an upper securement member having an adhesive on at least a portion of a bottom surface thereof, the upper securement member positioned over the stabilization anchor platform such that the stabilization anchor platform is positioned between the skin surface and the upper securement member, and wherein a perimeter of the upper securement member is larger than the perimeter of the stabilization anchor platform, such that a first portion of the upper securement member adheres to the skin surface and a second portion of the upper securement member adheres to the stabilization anchor platform.

5. The system of claim 4, wherein a second portion of the upper securement member adheres to a top surface of the stabilization anchor platform, via the adhesive on the bottom surface of the upper securement member.

6. The system of claim 4, wherein the upper securement member includes slots formed therein through which the extension tubing is routed.

7. The system of claim 1, wherein the attachment member comprises an external stabilization device having an adhesive on a bottom surface thereof that adheres to the skin surface, the external stabilization device including a cut-out portion formed therein configured to receive the stabilization anchor platform, and wherein the external stabilization device includes another adhesive that secures the stabilization anchor platform within the cut-out portion.

8. The system of claim 7, wherein a shape of the cut-out portion matches a shape of the stabilization anchor platform.

9. The system of claim 7, wherein the attachment member comprises both of the upper securement member and the external stabilization device.

10. The system of claim 1, wherein the attachment member comprises a pattern or layer of adhesive that adheres the stabilization anchor platform directly to the skin surface.

11. The system of claim 10, wherein the stabilization anchor platform comprises one or more pass-through features passing through the stabilization anchor platform from a front surface of the stabilization anchor platform to a back surface of the stabilization anchor platform, the one or more pass-through features configured to transfer and distribute adhesive applied thereto front the front surface to the back surface.

12. The system of claim 10, wherein the attachment member comprises both of the upper securement member and the pattern or layer of adhesive that adheres the stabilization anchor platform directly to the skin surface.

13. The system of claim 1, wherein the securement force is an adhering force of greater than 7 lbs, greater than 5 lbs, or greater than 3 lbs.

14. The system of claim 1, further comprising a clamp positioned along the extension tubing, wherein the stabilization anchor platform is positioned distal to the proximal access port and proximal to the clamp.

15. The system of claim 1, wherein the proximal access port comprises a needle-free connector.

16. The system of claim 15, wherein the force controlled release connector comprises a disconnect connector and a release feature coupled to the needle-free connector of the proximal access port, the release feature is engaged with the disconnect connector and configured to be disengaged from the disconnect connector when the predetermined separation force is applied to the disconnect connector or the needle-free connector of the proximal access port.

17. The system of claim 1, further comprising a secondary stabilization anchor platform configured to stabilize the catheter device.

18. The system of claim 1, wherein the extension tubing is integral with the catheter adapter, as part of an integrated catheter system.

19. The system of claim 1, wherein the extension tubing and the catheter connector are coupleable to a catheter hub connected to the catheter, as part of a non-integrated catheter system.

20. A method for securing a catheter system to the skin surface of a patient, the method comprising:
providing a catheter system having:
a catheter device for receiving a catheter and including a side port in fluid communication with the catheter, the catheter device comprising a catheter adapter or catheter connector, extension tubing in fluid communication with the side port of the catheter device, the extension tubing having a proximal access port coupled to a proximal end portion thereof,
a force controlled release connector coupled to the proximal access port, wherein at least a portion of the force controlled release connector is configured to separate from the proximal access port when a predetermined separation force is applied to the force controlled release connector,
a stabilization anchor platform spaced from the catheter device and positioned along the extension tubing, distal from the force controlled release connector, and
an attachment member for securing the stabilization anchor platform relative to a skin surface of a patient;
adhering the stabilization anchor platform in place relative to the skin surface via the attachment member; and
wherein, the attachment member retains the stabilization anchor platform in place relative to the skin surface with a securement force that is greater than the predetermined separation force that causes the force controlled release connector to separate from the proximal access port.
